(19) United States
(12) Patent Application Publication
(10) Pub. No.: US 2020/0114014 A1
(43) Pub. Date: Apr. 16, 2020

(54) Novel Alpha-Hydroxy Carboxylic Acid and Derivatives and Other GRAS-Based Amide and Imide Prodrugs of Amphetamine Compounds and Uses Thereof

(71) Applicant: John K. Thottathil, Mundelein, IL (US)

(72) Inventor: John K. Thottathil, Mundelein, IL (US)

(21) Appl. No.: 16/603,874

(22) PCT Filed: Apr. 10, 2018
(86) PCT No.: PCT/US18/26819

§ 371 Date: Oct. 9, 2019

Related U.S. Application Data
(60) Provisional application No. 62/484,022, filed on Apr. 11, 2017.

(51) Int. Cl. A61K 47/54; A61K 31/137; A61K 31/445

(52) U.S. Cl. CPC A61K 47/542; A61K 31/445; A61K 31/137

(57) ABSTRACT

The invention describes pharmaceutical compounds and compositions comprised of a ligand attached to stimulants (CNS drugs) in a manner that substantially decreases or deters the potential for stimulants abuse, addiction, illicit and illegal use, and overdose. These compounds and compositions find particular use in providing an abuse-resistant alternative treatment for certain disorders, such as attention deficit hyperactivity disorder (ADHD), ADD, narcolepsy, and obesity. When delivered at the proper dosage, the pharmaceutical composition provides therapeutic activity similar to that of the parent active agent.

### CROSS REFERENCE TO RELATED APPLICATIONS

This application claims the benefit of U.S. Provisional Application No. 62/484,022, filed Apr. 11, 2017.

### FIELD OF INVENTION

The present invention relates to pharmaceutical compounds, compositions, and methods of using chemical moieties that are generally recognized as safe (GRAS), which are attached to CNS stimulants such as amphetamine compounds. These chemical moieties are monomers, homo- and hetero-oligomers, of alpha-hydroxy carboxylic acids and their chemical derivatives. In one embodiment, the invention relates to amphetamine and methylphenidate prodrugs comprising these stimulants covalently bound to the chemical moieties. These inventions provide a variety of beneficial effects, particularly a substantial decrease in the potential of stimulants such as amphetamine and methylphenidate to cause overdose or to be abused. Some embodiments of the invention provide therapeutic activity similar to that of unmodified stimulants at typical dosage ranges, but when delivered at higher doses, the potential for overdose is reduced due to decreased bioavailability of the stimulants, especially when taken by non-approved routes, as compared to stimulants that are administered by the approved oral route and delivered in a non-conjugated form. Additionally, these prodrugs may be designed to provide fast or slow release of stimulants depending on their standard use for various ailments.

### BACKGROUND OF THE INVENTION

Amphetamines stimulate the central nervous system (CNS) and have been used medicinally to treat various disorders including attention deficit hyperactivity disorder (ADHD), obesity, and narcolepsy. Potent CNS stimulants have been used for several decades as a drug treatment given either alone or as an adjunct to behavioral therapy in children with ADHD. While methylphenidate (Ritalin®, Focalin®) has been the most frequently prescribed stimulant, the prototype of the class amphetamine (alpha-methyl phenethylamine) has been used all along and increasingly so in recent years (Bradley C, Bowen M, “Amphetamine (benzedrine) therapy of children's behavior disorders” American Journal of Orthopsychiatry 11: 92-103 (1941). Amphetamine drugs (Vyvanse®, Adderall®) remain a popular and effective medication for ADHD.

The amphetamine compounds, and their derivatives and analogs, are subject to abuse due to their stimulating effects. A user can become dependent over time on these drugs and their psychological effects, even when the drugs are used for legitimate therapeutic purposes. Legitimate amphetamine users that develop drug tolerances are especially susceptible to becoming accidental addicts as they increase dosing in order to counteract their increased tolerance of the prescribed drugs. Additionally, it is possible for individuals to inappropriately self-administer higher than prescribed quantities of the drug or to alter either the product or the route of administration (e.g., inhalation (snorting), IV injection, and smoking), potentially resulting in immediate release of the active drug in quantities larger than prescribed. When taken at higher than prescribed doses, these stimulants can cause temporary feelings of exhilaration and increased energy and mental alertness.

Recent developments in the abuse of prescription drug products increasingly raise concerns about the abuse of stimulants prescribed for ADHD. The National Survey on Drug Use and Health (NSDUH) estimates that in 2003, 1.2 million Americans aged 12 and older abused stimulants, such as amphetamines. The high abuse potential has earned amphetamines Schedule II status according to the Controlled Substances Act (CSA). Schedule II classification is reserved for those drugs that have accepted medical use but have the highest potential for abuse.

Sustained release formulations of amphetamines, e.g., Adderall XR®, have an increased abuse liability relative to the single dose tablets because each tablet of the sustained release formulation contains higher concentration of amphetamine. It may be possible for substance abusers to obtain a high dose of amphetamine with rapid onset by crushing the tablets into powder and snorting it or by dissolving the powder in water and injecting it intravenously. Sustained release formulations may also provide uneven release of the drug in the patient's bloodstream.

Accidental and intentional overdose with prescription and/or over-the-counter (OTC) drugs is a serious health problem that is associated with thousands of fatalities every year. Stimulant drug overdose is a significant and growing problem associated with drug abuse, but overdoses also occur accidentally (e.g., when a child obtains and ingests a stimulant drug, such as Adderall®, Ritalin®, or Focalin®), or intentionally (e.g., when related to suicide attempts). Accidental overdose can also commonly occur when unusually potent batches of illicit stimulants are ingested by drug addicts or other abusers.

The need exists for additional amphetamine and other stimulant compounds that are abuse resistant and which provide sustained release and sustained therapeutic effect.

Researchers and the pharmaceutical industry have sought to prevent the potential harmful effects of these types of drug overdose by creation of various drug formulations. To cite an example, extended-release methylphenidate (Concerta®) has been formulated in a paste that can preclude administration by snorting or injection. Opioids, another class of highly abused drugs have been combined with opioid antagonists during formulation. Such formulations are designed to counteract an oral stimulant if the formulation is disrupted (e.g., crushed) prior to oral administration, or if the drug is administered parenterally (e.g., injected intravenously). An example is the drug Embeda® where the opioid drug morphine is co-formulated with the antagonist naltrexone in a sequestered fashion. Other compositions have been coated with emetic agents in quantities that—if administered in moderation as intended, no emesis occurs; however, if excessive amounts are ingested, emesis is induced to prevent overdose. However, such methods, as well as conventional controlled-release formulations, are often ineffective and can be circumvented.

Consequently, improved methods are urgently needed to make pharmaceutically effective stimulant compounds, along with compositions and methods of using such compounds, to reduce the potential for overdose and to reduce or deter stimulant substance abuse while maintaining intended CNS therapeutic utility. Potentially useful compounds may also prevent—or substantially diminish or delay—uptake into the brain if the compounds are administered by routes other than approved oral administration.

Prodrug chemistry has been tried and met with limited success in the case of amphetamine as demonstrated by Vyvanse.

Ideally, a prodrug moiety and its linkage to a particular stimulant would be cleaved at an appropriate rate and site, which would then release the active stimulant compound into the blood and provide the intended CNS benefit. There remains a critical need for the treatment of CNS diseases with stimulants using products that retain all their pharmacological advantages but sharply reduce their principal limitations, including variable bioavailability after oral dosing, overdose, misuse, illegal/illicit use and product tampering.

### SUMMARY

Provided are pharmaceutical compounds, compositions, and methods of using such compounds and compositions. Also provided are methods of using chemical moieties that are generally recognized as safe (GRAS), which are attached to stimulant molecules including amphetamine and methylphenidate. These chemical moieties are monomers, homo- and hetero-oligomers of alpha-hydroxy carboxylic acids, and their chemical derivatives. The compounds may provide a substantial decrease in the potential of stimulants (e.g., amphetamine, methylphenidate) to cause overdose or to be abused. In some embodiments these stimulant prodrug conjugates provide therapeutic activity which is similar to that of the unmodified parent drug when delivered at typical dosage ranges. However, when delivered at higher doses the potential for overdose is reduced as compared to conventional non-conjugated stimulant due to decreased bioavailability of the stimulant, especially when taken by non-approved non-oral routes. Additionally, the prodrugs may be designed to provide fast or slow release of the stimulant depending on its standard use for chronic or acute causes.

The drugs/molecules that are contemplated in this invention include all the stimulant drugs including but not limited to, amphetamine and methylphenidate.

### DETAILED DESCRIPTION OF THE INVENTION

A first aspect of the invention relates to changing the pharmacokinetic and pharmacological properties of stimulant drugs such as amphetamine and methylphenidate through covalent modification of these stimulants using alpha-hydroxy carboxylic acid and derivatives and other generally recognized as safe (GRAS)-based moieties to produce prodrugs of the stimulants. Covalent attachment of a chemical moiety—specifically, a moiety derived from alpha-hydroxy carboxylic acid and derivatives and other GRAS-based reagents as monomers and oligomers (homo and hetero oligomers)—to stimulants may change one or more of the following properties of stimulants: the rate of absorption; extent of absorption and distribution within the body; metabolism and drug elimination (i.e., ADME pharmacokinetic properties). As such, the alteration of one or more of these characteristics may be designed to provide fast or slow release of the parent drug, depending on need for relief of chronic versus acute CNS diseases. Additionally, alteration of one or more of these characteristics may reduce the previously noted side-effects associated with stimulants. In turn, these alterations may diminish or deter abuse potential. The oligomers formed from alpha-hydroxy carboxylic acid and derivatives can be homo- or hetero-‘mers’ and can be either linear or branched ‘mers’. The hetero ‘mers’ can be cross linked with other GRAS reagents, such as other alpha-hydroxy carboxylic acid, amino acid and dicarboxylic acids including, but not limited to, fumaric acid, maleic acid and succinic acid.

The stimulant prodrugs may also prevent abuse by exhibiting stability under conditions that are likely to be employed by chemists who may illicitly attempt to release the stimulant compound from its attached group. The stimulant prodrugs may further prevent abuse by exhibiting reduced bioavailability when administered via parenteral routes, particularly by intravenous, intranasal, or inhalation (“smoking”) routes that are often employed in illicit use. Thus, the stimulant prodrugs may reduce the desired euphoric effect associated with stimulant abuse. Thus, the stimulant prodrug may prevent, deter, or reduce abuse potential and overdose when the stimulant prodrug is used in an unapproved manner (e.g., ingestion at a higher dose or non-oral administration).

Stimulant prodrugs of the present invention may be depicted as the structure shown by Formula A where “STI” represents the stimulant compound (e.g., an amphetamine compound) and “X” represents the prodrug component that is chemically/covalently attached to the stimulant “STI”. The prodrug component “X” can be any moiety that decreases the pharmacological activity of the stimulants while bound to the parent molecule, as compared to unbound (free) stimulants.

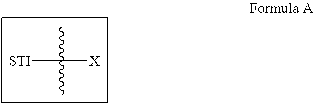

Specifically, certain embodiments of the stimulant prodrugs of the present invention may be depicted as Formula B where “STI” represents amphetamine and Formula C where “STI” represents methylphenidate and moiety X represents the prodrug component. Formula

B is the amphetamine prodrug in which the prodrug moiety X is chemically/covalently attached to the amine group of amphetamine as the amide. Formula C is the methylphenidate prodrug in which the prodrug moiety X is chemically/covalently attached to the amine group of methylphenidate as the amide.

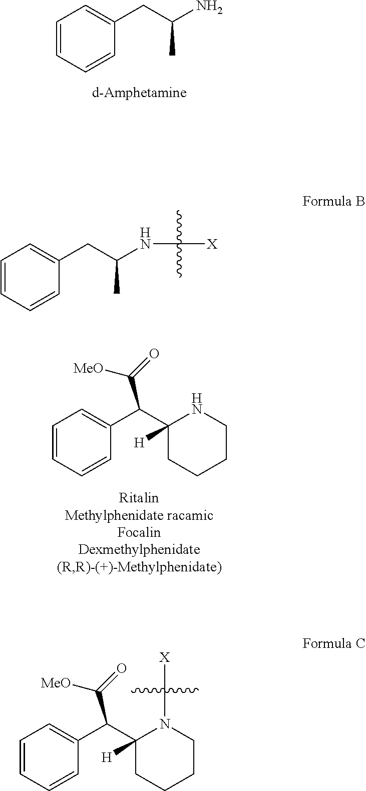

Stimulant prodrugs of the present invention may also be depicted as the structure shown by Formula D where “Y” is a ligand which forms the prodrug moiety as a five-membered, six-membered, or seven-membered imide ring formed around the amphetamine amine functional group. Y represents the remaining structure of the imide ring, as discussed in more detail below.

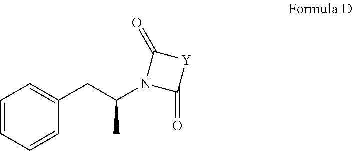

The amphetamine compounds of the present invention can be any of the sympathomimetic phenethylamine derivatives which have central nervous system stimulant activity such as amphetamine, or any derivative, analog, or salt thereof. Exemplary amphetamines include, but are not limited to, amphetamine, methamphetamine, methylphenidate, p-methoxyamphetamine, methylenedioxyamphetamine, 2,5-dimethoxy-4-methylamphetamine, 2,4,5-trimethoxyamphetamine, and 3,4-methylenedioxymethamphetamine, N-ethylamphetamine, fenethylline, benzphetamine, and chlorphentermine as well as the amphetamine compounds of Adderall®; actedron; actemin; adipan; akedron; allodene; alpha-methyl-(±)-benzeneethanamine; alpha-methylbenzeneethanamine; alpha-methylphenethylamine; amfetamine; amphate; anorexine; benzebar; benzedrine; benzyl methyl carbinamine; benzolone; beta-amino propylbenzene; beta-phenylisopropylamine; biphetamine; desoxynorephedrine; dietamine; DL-amphetamine; elastonon; fenopromin; finam; isoamyne; isomyn; mecodrin; monophos; mydrial; norephedrane; novydrine; obesin; obesine; obetrol; octedrine; oktedrin; phenamine; phenedrine; phenethylamine, alpha-methyl-; percomon; profamina; profetamine; propisamine; racephen; raphetamine; rhinalator, sympamine; simpatedrin; simpatina; sympatedrine; and weckamine. Preferred amphetamines include methamphetamine, methylphenidate, and amphetamine. As used herein, reference to an “amphetamine compound,” “amphetamines” or “stimulant” includes any of the foregoing sympathomimetic phenethylamine derivatives, i.e., the term encompasses the “class” of amphetamines. Reference to “amphetamine” means the specific stimulant molecule amphetamine.

The amphetamines of the present invention (including methylphenidate) can have any stereogenic configuration, including both dextro- and levo-isomers, racemates, and mixtures of the two isomers of varying ratios. The dextro-isomers, particularly dextroamphetamine and (R, R)-(+)-methylphenidate are preferred.

Alpha-hydroxy carboxylic acids and other GRAS-based monomers used to make the monomer-based and oligomer-based stimulant prodrugs, Formula B, Formula C, and Formula D are depicted below.

It should be emphasized that the following chemical moieties represent non-limiting examples of alpha-hydroxy carboxylic acids and other GRAS-based monomers used to make the monomer-based and oligomer-based stimulant prodrugs of the present invention:

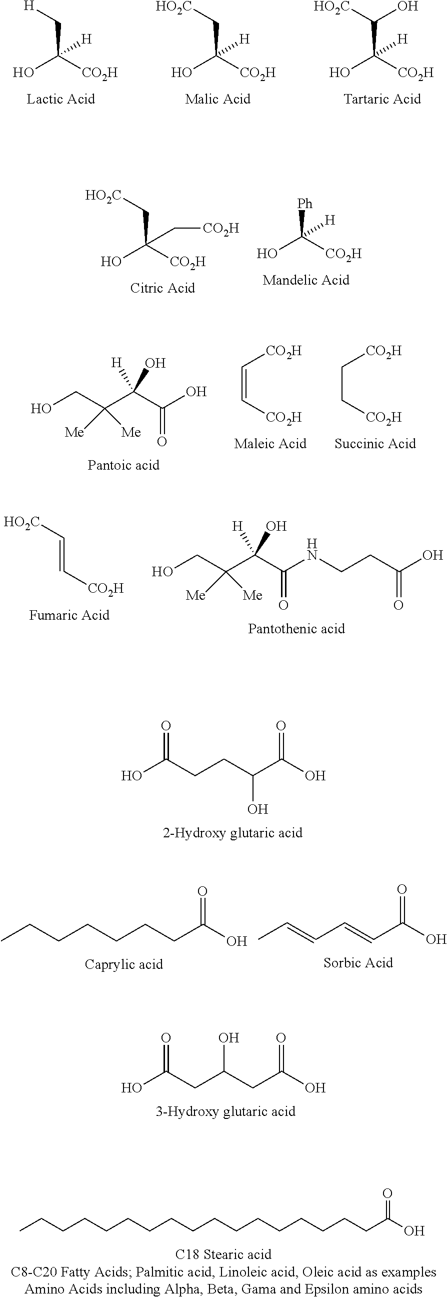

The alpha-hydroxy carboxylic acids represented here for use in the invention include the naturally occurring (L)-isomers, the non-natural (D)-isomers, mixtures of (L) and (D) isomers, racemates and mixtures of diastereomers, and meso-isomers. The term “alpha-hydroxy carboxylic acid” as used herein is intended to encompass any or all of the foregoing variants.

The amino acids represented here for use in the invention include both natural and non-natural amino acids, the naturally occurring (L)-isomers, the non-natural (D)-isomers, mixtures of (L) and (D) isomers, racemates and mixtures of diastereomers. The term “amino acid” as used herein is intended to encompass any or all of the foregoing variants. When reference is made to a “side chain” of an amino acid, it is intended that the side chain may be the side chain of any of the foregoing types of amino acid.

The amino acids represented here for use in the invention also include alpha amino acids, beta amino acids, gamma amino acids, and epsilon amino acids (remote amino group relative to the carboxyl group), and di-carboxylic acid amino acids such as aspartic acid and glutamic acid. The term “amino acid” as used herein is intended to encompass any or all of the foregoing variants. When reference is made to a “side chain” of an amino acid, it is intended that the side chain may be the side chain of any of the foregoing types of amino acid.

The fatty acids represented here for use in the invention include long-chain carboxylic acids, ranging in carbon lengths between eight carbons (C8) to twenty carbons (C20). These fatty acids may be linear or branched and either saturated or unsaturated. In the case of unsaturated fatty acids, both cis- and trans-isomers (Z and E isomers) are contemplated. The term “fatty acid” as used herein is intended to encompass any or all of the foregoing.

The alpha-hydroxy carboxylic acids and other GRAS-based monomers represented here are used to make the monomer-based and oligomer-based stimulant prodrugs of Formula B, Formula C and Formula D.

Di-carboxylic acids such as malic acid, tartaric acid, citric acid, hydroxy glutaric acids (both 2-hydroxy and 3-hydroxy), succinic acid, maleic acid, and di-carboxylic acid amino acids such as aspartic acid, glutamic acid may be used to make the monomer-based and oligomer-based stimulant prodrugs of Formula D.

In one embodiment of the present invention, the prodrug component X may be represented as,

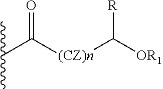

wherein,
CZ =CH2, or CHOR1,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Methyl (Me), Phenyl (Ph), CH2COR2, CHOR1COR2, or COR2 (when n is not zero), where R2=OH, an ester formed by the hydroxyl group of another alpha-hydroxy acid or an amide formed by the amine group of an amino acid, and,
n is an integer selected from 0 to 2.

In another embodiment of the present invention, the prodrug component X may be represented as,

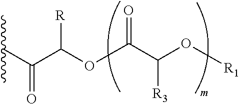

wherein,
R1=H, an acyl linkage of a fatty acid, an acyl linkage of an alpha-hydroxy acid, an acyl linkage of an amino acid or an acyl linkage of a dicarboxylic acid including, but not limited to, fumaric acid, maleic acid and succinic acid, and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

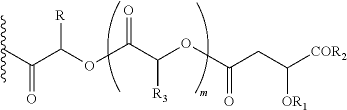

wherein,
R1=H, an acyl linkage of a fatty acid, an acyl linkage of an alpha-hydroxy acid, an acyl linkage of an amino acid or an acyl linkage of a dicarboxylic acid including, but not limited to, fumaric acid, maleic acid and succinic acid, and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

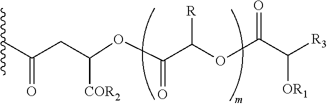

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

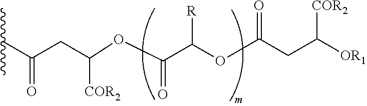

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,

R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

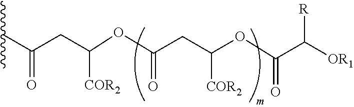

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

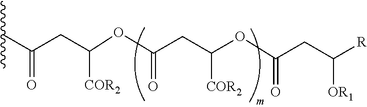

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

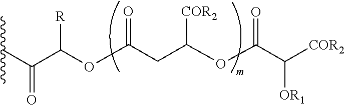

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4.

In another embodiment of the present invention, the prodrug component X may be represented as,

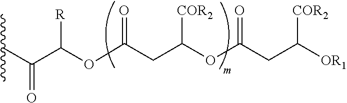

wherein,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R=Me, Ph, CH2COR2, or CHOR1COR2, where R2=OH or is an ester formed by the hydroxyl group of another alpha-hydroxy acid or is an amide formed by the amine group of an amino acid, and,
m is an integer selected from 0 to 4

In another embodiment of the present invention, the prodrug component X may be represented as,

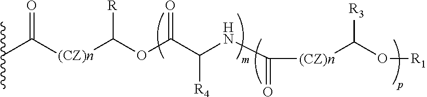

wherein,
CZ=CH2, or CHOR1;
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
R4 is the side chain of an amino acid, and,
m is an integer selected from 0 to 4, and,
n is an integer selected from 0 to 2, and,
p is an integer selected from 0 to 1.

In another embodiment of the present invention, the prodrug component X may be represented as,

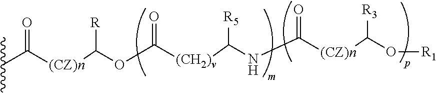

wherein,
CZ=CH2, or CHOR1;
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
R5=H, or COR2, where R2=OH or is an ester formed by the hydroxyl group of another alpha-hydroxy acid or is an amide formed by the amine group of an amino acid, or is an alkyl ester (O-alkyl, wherein the alkyl group is a 1-4 carbon linear or branched, saturated or non-saturated alkyl group), and,
m is an integer selected from 0 to 4, and,
n is an integer selected from 0 to 2, and,
p is an integer selected from 0 to 1, and,
v is an integer selected from 0 to 5.

In another embodiment of the present invention, the prodrug component X may be represented as,

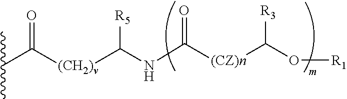

wherein,
CZ=CH2, or CHOR1;
R1=H, an acyl linkage of a fatty acid, an acyl linkage of an alpha-hydroxy acids, an acyl linkage of an amino acid, or an acyl linkage of a dicarboxylic acid including, but not limited to, fumaric acid, maleic acid and succinic acid, and,
R3=Me, Ph, CH2COR2, or CHOR1COR2, where R2=OH or is an ester formed by the hydroxyl group of another alpha-hydroxy acid or is an amide formed by the amine group of an amino acid, and
R5=H, or COR2, where R2=OH or is an ester formed by the hydroxyl group of another alpha-hydroxy acid or is an amide formed by the amine group of an amino acid, or is an alkyl ester (O-alkyl, wherein the alkyl group is a 1-4 carbon linear or branched, saturated or un-saturated alkyl group), and,
m is an integer selected from 0 to 4, and,
n is an integer selected from 0 to 2, and,
v is an integer selected from 0 to 5.

In another embodiment of the present invention, the prodrug component X may be represented as,

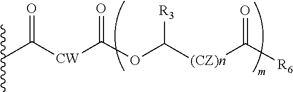

wherein,
CW=(CH2)q, or CH═CH (both E and Z isomers),
CZ=CH2, or CHOR1;
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R3=Me, Ph, CH2COR2, or CHOR1COR2, where R2=OH or is an ester formed by the hydroxyl group of another alpha-hydroxy acid or is an amide formed by the amine group of an amino acid, and,
R6=OH or is an ester formed by the hydroxyl group of another alpha-hydroxy acid or is an amide formed by the amine group of an amino acid, or is an alkyl ester (O-alkyl, wherein the alkyl group is a 1-4 carbon linear or branched, saturated or non-saturated alkyl group), and,
m is an integer selected from 0 to 4, and,
n is an integer selected from 0 to 2, and,
q is an integer selected from 2 to 6.

In another embodiment of the present invention, the prodrug component X may be represented as,

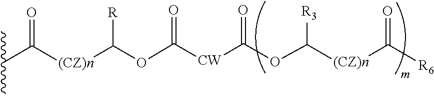

wherein,
CW=(CH2)q, or CH═CH (both E and Z isomers),
CZ=CH2, or CHOR1;
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R and R3 can be same or different, and,
R and R3=Me, Ph, CH2COR2, or CHOR1COR2, where R2=OH or is an ester formed by the hydroxyl group of another alpha-hydroxy acid or is an amide formed by the amine group of an amino acid, and,
R6=OH or is an ester formed by the hydroxyl group of another alpha-hydroxy acid or is an amide formed by the amine group of an amino acid, or is an alkyl ester (O-alkyl, wherein the alkyl group is a 1-4 carbon linear or branched, saturated or unsaturated alkyl group), and
m is an integer selected from 0 to 4, and,
n is an integer selected from 0 to 2, and,
q is an integer selected from 2 to 6.

In another embodiment of the present invention, the prodrug components X may be represented as,

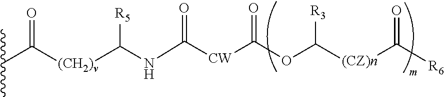

wherein,
CZ=CH2, or CHOR1;
CW=(CH2)q, or CH═CH, (including both E and Z isomers), and,
R3=Me, Ph, CH2COR2, or CHOR1COR2, where R2=OH or is an ester formed by the hydroxyl group of another alpha-hydroxy acid or is an amide formed by the amine group of an amino acid, and,
R5=H, or COR2, where R2=OH or is an ester formed by the hydroxyl group of another alpha-hydroxy acid or is an amide formed by the amine group of an amino acid, or is an alkyl ester (O-alkyl, wherein the alkyl group is a 1-4 carbon linear or branched, saturated or un-saturated alkyl group), and,
R6=OH or is an ester formed by the alcohol (OH) group of an alpha-hydroxy acid or is an amide formed by the amine group of an amino acid, or is an alkyl ester (O-alkyl, wherein the alkyl group is a 1-4 carbon linear or branched, saturated and unsaturated alkyl group), and,
m is an integer selected from 0 to 4, and,
n is an integer selected from 0 to 2, and,
q is an integer selected from 2 to 6, and,
v is an integer selected from 0 to 6.

In another embodiment of the present invention, the prodrug component X may be represented as,

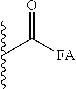

wherein,
FA is C8 to C20 saturated or unsaturated fatty acid including sorbic acid, stearic acid, oleic acid, palmitic acid, and linoleic acid. The fatty acids may be linear or branched chain acids, or a combination thereof; and in the case of unsaturated fatty acids, they may be cis- or trans-isomers (Z and E isomers).

In another embodiment of the present invention, the amphetamine compound prodrug is represented as the imide structure, Formula D wherein the prodrug moiety is a five-membered, six-membered or seven-membered imide ring formed around the amphetamine amine functional group. More specifically,

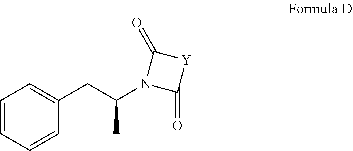

### Wherein,

Y=(CH2)t, CH═CH, CH2—CH(OR7), CH(OR7)—CH(OR8), CH2C(OR7)(CH2COR9), CH2—C(OR7)(COR9)—CH2, CH2—CH(OR7)—CH2, CH2—CH2—CH(OR7), CH2—CH(NR10), CH2—CH2—CH(NR10), or CH2—CH(NR10)—CH2, and t is an integer selected from 0 to 4. R7-R10 are defined below.

Specific examples of compounds according to Formula D may be depicted as Formulae E-P:

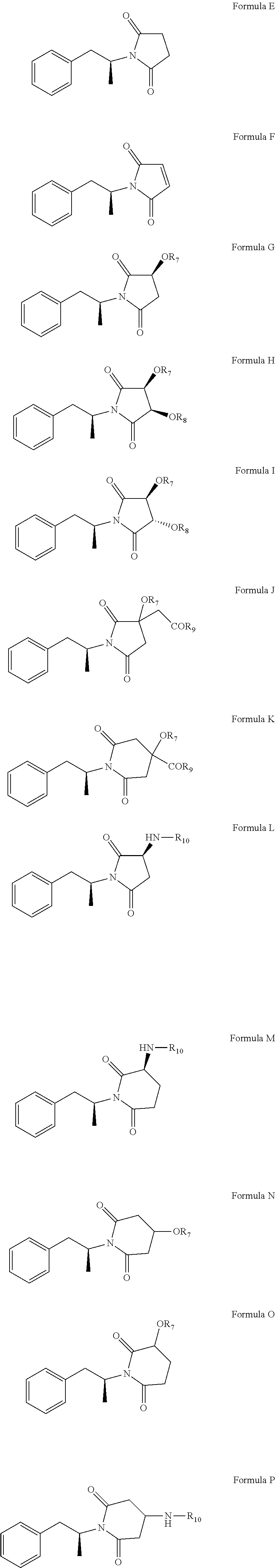

### Wherein,

R7 and R8 are each independently H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R9=OH, or an ester formed by the hydroxyl group of another alpha-hydroxy acid or an amide formed by the amine group of an amino acid, and,
R10=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid.

It should be emphasized that the five-membered imide ring formed with amphetamine nitrogen belongs to the class of compounds such as: succinimide (formula E), malei-imide (formula F). malic acid imide (formula G, both isomers), tartaric acid imide (formula H, formula I, RR, SS, and meso-isomers), citric acid imide (formula J), aspartic acid imide (formula L).

It should be also be emphasized that the six-membered imide ring formed with amphetamine nitrogen belongs to the class of compounds such as: citric acid imide (formula K), glutamic acid imide (formula M), 2-hydroxy glutaric acid imide (formula O), 3-hydroxy glutaric acid imide (formula N), 3-amino glutaric acid imide (formula P).

Upon prodrug cleavage, these amphetamine prodrugs of Formula B and Formula D will revert back to the original amphetamine molecule with the amine group present intact.

Upon prodrug cleavage, the methylphenidate prodrug of Formula C will revert back to the original methylphenidate molecule with the amine group present intact.

The alpha-hydroxy carboxylic acid and its homo and hetero oligomers (with another alpha-hydroxy carboxylic acid) referred to in this invention should be understood to be covalently bound via a hydroxy group on the alpha-hydroxy carboxylic acid or on the oligomer to another carbonyl (originally part of a carboxyl group of another alpha-hydroxy carboxylic acid, or to another carbonyl of the carboxyl group of the amino acid, or to one carbonyl of the carboxyl group of a dicarboxylic acid (e.g., succinic acid, maleic acid, fumaric acid), while the carboxyl group from the initial alpha-hydroxy carboxylic acid is attached to the stimulant.

If the initial carboxyl group that is attached to the amphetamine compound amine group referred to in this invention is from a dicarboxylic acid (e.g. malic acid, tartaric acid, citric acid, hydroxy-glutaric acid, succinic acid), it should be understood that the originally formed amide can function as ligand formation for structures of Formula B type (for amphetamine) and structures of Formula C type (for methylphenidate).

If the initial carboxyl group that is attached to the amphetamine compound amine group referred to in this invention is from a dicarboxylic acid (e.g. malic acid, tartaric acid, citric acid, hydroxy-glutaric acid, succinic acid), it should also be understood that the second carboxyl group present in the original di-carboxylic acid can swing around and cyclize with the nitrogen atom present in amphetamine forming five and six membered imide structures of

Formulae E-K and Formulae N-O.

If the initial carboxyl group that is attached to the amphetamine compound amine group referred to in this invention is from an acidic amino acid (e.g. aspartic acid, glutamic acid), it should be understood that the second carboxyl group present in the amino acid can swing around and cyclize with the nitrogen atom present in amphetamine forming five and six membered imide structures of Formula L, Formula M and Formula P.

If the initial carboxyl group that is attached to the stimulant referred to in this invention is from an acidic amino acid (e.g. aspartic acid, glutamic acid), it should be understood that the amino group of the said amino acid may be bound via a covalent bond as the amide with the carboxyl group on the alpha-hydroxy carboxylic acid or the oligomer carbonyl (originally part of a carboxyl group of the alpha-hydroxy carboxylic acids) or to one carbonyl of the carboxyl group of a dicarboxylic acid (e.g., succinic acid, maleic acid, fumaric acid), or to the carbonyl of the carboxyl group of the fatty acids.

It should also be understood that If the initial carboxyl group that is attached to the stimulants referred to in this invention is from alpha-hydroxy carboxylic acids and its homo and hetero oligomers (with another alpha-hydroxy carboxylic acid) to form structures of Formula B-D, the ensuing hydroxyl group may be capped as its ester by fatty acids.

It should also be understood that If the initial carboxyl group that is attached to the stimulant referred to in this invention is from alpha-hydroxy carboxylic acids and its homo and hetero oligomers (with another alpha-hydroxy carboxylic acid) to form structures of Formula B-D, the terminal hydroxyl group may be capped as its ester by dicarboxylic acids (e.g., succinic acid, maleic acid, fumaric acid).

It should also be understood that If the initial carboxyl group that is attached to the stimulant referred to in this invention is from alpha-hydroxy carboxylic acids and its homo and hetero oligomers (with another alpha-hydroxy carboxylic acid) to form structures of Formula B-D, the terminal hydroxyl group may be capped as its ester by amino acids

In another embodiment of the present invention, when the covalently modified stimulant (e.g., amphetamine, methylphenidate) is provided in oral dosage form (e.g., a tablet, capsule, caplet, liquid dispersion, etc.) it has increased resistance to manipulation. For instance, crushing of a tablet or disruption of a capsule does not substantially increase the rate and amount of stimulant absorbed when compositions of the invention are ingested.

In another embodiment of the present invention, when the stimulant covalently bound to the prodrug moiety is provided in oral dosage form: for example a tablet, capsule, caplet or other formulation it is resistant to generation of stimulant by physical manipulation such as crushing.

Another embodiment of the present invention provides stimulant prodrug conjugates as a composition or method for treating CNS diseases in patients. It should be noted that different conjugates maybe be utilized to treat acute versus chronic conditions.

Another embodiment of the present invention is a composition or method for a sustained-release stimulant comprising a covalently bonded stimulant conjugate, wherein said conjugate provides release of stimulant at a rate where the level of stimulant is within the therapeutic range, but below toxic levels, over an extended period of time (e.g., 8-24 hours or greater).

Another embodiment of the present invention is a composition or method for reducing variability in bioavailability, or preventing a toxic release of stimulant, comprising the stimulant covalently bonded to the prodrug moiety, wherein said bound stimulant maintains a steady-state plasma release curve, which provides therapeutically effective bioavailability but prevents spikes or sharp increases in blood concentrations compared to unbound stimulant when given at doses exceeding those that are within the therapeutic range of the stimulant.

Another embodiment of the invention is a composition or method for preventing a Cₘₐₓ spike for stimulant while still providing therapeutically effective bioavailability curve comprising stimulant which has been covalently bonded to the prodrug moiety.

Another embodiment of the present invention is a method for reducing or preventing abuse related to the euphoric effect of a pharmaceutical stimulant composition, comprising consuming said composition, wherein said composition comprises a prodrug moiety covalently attached to stimulant, such that the pharmacological activity of stimulant is substantially decreased when the composition is used in a manner inconsistent with approved instructions or in a manner that substantially increases the potential of overdose.

Other embodiments of the present invention are methods wherein said pharmaceutical composition is adapted solely for oral administration, and wherein said stimulant is resistant to release from said prodrug moiety when the composition is administered parenterally (e.g., intranasally. intravenously. etc.). Preferably, said stimulant would be preferentially released from said chemical moiety primarily in the presence of acid and/or enzymes present in the stomach or intestinal tract, respectively.

In another embodiment of the present invention, the covalently bonded stimulant prodrug may also be in a pharmaceutically acceptable salt form. Pharmaceutically acceptable inorganic and organic acid addition salts are known in the art. Exemplary salts include, but are not limited to, hydrobromide, hydrochloride, hydroiodide, benzoate, bisulfate, tartrate, bitartrate, edetate, edisylate, estolate, esylate, ethanesulfonate, lactate, malate, maleate, mandelate, methanesulfonate, phosphate, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, borate, butyrate, calcium edetate, camphorate, camphorsulfonate, citrate, clavulariate, cyclopentanepropionate, digluconate, dodecylsulfate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydroxynaphthoate, isothionate, lactobionate, laurate, laurylsulphonate, mucate, naphthylate, napsylate, nicotinate, N-methylglucamine ammonium salt, oleate, palmitate, pamoate, pantothenate, pectinate, phosphateldiphosphate, pivalate, polygalacturonate, propionate, p-toluenesulfonate, saccharate, salicylate, stearate, subacetate, succinate, sulfate, sulfosaliculate, suramate, tannate, teoclate, tosylate, triethiodide, undecanoate, and valerate salts, and the like.

The term “amino acid” refers to one of twenty-two amino acids used for protein biosynthesis, as well as other amino acids that can be incorporated into proteins during translation. Such amino acids can be a natural amino acid, such as glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine, histidine and beta alanine, or non-natural amino acids and alpha amino acids, beta amino acids, gamma amino acids, and epsilon amino acids (e.g., the amino group is remote relative to the carboxyl group).

The present invention also provides methods for providing, administering, prescribing, or consuming a stimulant prodrug. The invention also provides pharmaceutical compositions comprising a stimulant prodrug. The formulation of such a pharmaceutical composition can optionally enhance or achieve the desired release profile.

In one embodiment, the invention provides methods for treating a patient comprising administering a therapeutically effective amount of a stimulant (amphetamine, methylphenidate) prodrug, i.e., an amount sufficient to prevent, ameliorate, and/or eliminate the symptoms of a disease. These methods can be used to treat any disease that may benefit from amphetamine-type drugs including, but not limited to: attention deficit disorders, e.g., ADD and ADHD, and other learning disabilities; obesity; Alzheimer's disease, amnesia, and other memory disorders and impairments; fibromyalgia; fatigue and chronic fatigue; depression; epilepsy; obsessive compulsive disorder (OCD); oppositional defiant disorder (ODD); anxiety; resistant depression; stroke rehabilitation; Parkinson's disease; mood disorder; schizophrenia; Huntington's disorder; dementia, e.g., AIDS dementia and frontal lobe dementia; movement disfunction; apathy; Pick's disease; Creutzfeldt-Jakob disease, sleep disorders, e.g., narcolepsy, cataplexy, sleep paralysis, and hypnagogic hallucinations; conditions related to brain injury or neuronal degeneration, e.g., multiple sclerosis, Tourette's syndrome, and impotence; and nicotine dependence and withdrawal. Preferred indications include ADD, ADHD, narcolepsy, and obesity, with ADHD being most preferred.

In a further embodiment of the present invention, non-limiting examples of stimulant prodrugs of amphetamine compounds are shown in Formulae 1-90 and formulae E-P. In these formulae, it should be noted that while no salt forms have been depicted, all the formulae compounds can be prepared as their pharmaceutically acceptable salts, as previously described. It should also be noted that in formulae 1-90, “STI” represent the stimulant and the prodrug component X is chemically/covalently attached to the stimulant amphetamine compound “STI”. In these formulae 1-90, it should also be noted that the stimulant represented here include non-limiting examples of amphetamine and methylphenidate. The point of covalent attachment of the ligands to these stimulants is at the nitrogen atom in the molecule and it is an amide bond. In the case of Formulae E-P, it should be noted that R7, R8 and R10 can independently either H (hydrogen) or any of the ligands that are attached to STI in formulae 1-90, and R9 is either OH, or is an ester formed by the hydroxyl group of another alpha-hydroxy acid or an amide formed by the amine group of an amino acid.

Non-limiting examples of stimulant prodrugs formulae 1-90 and formulae E-P are:

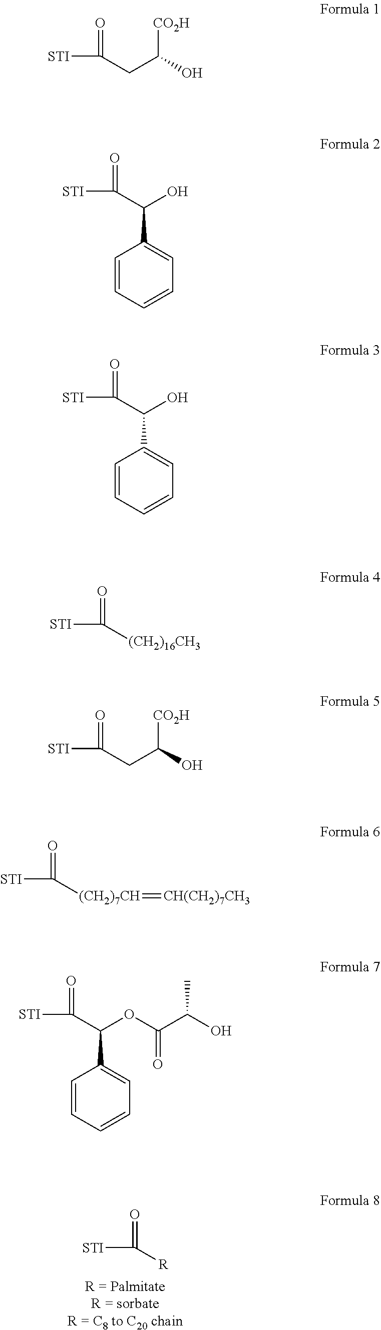

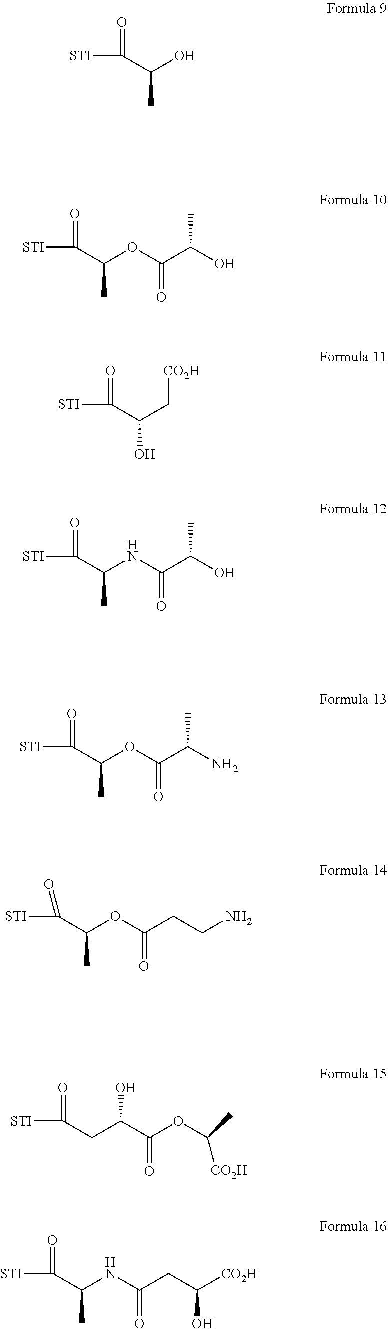

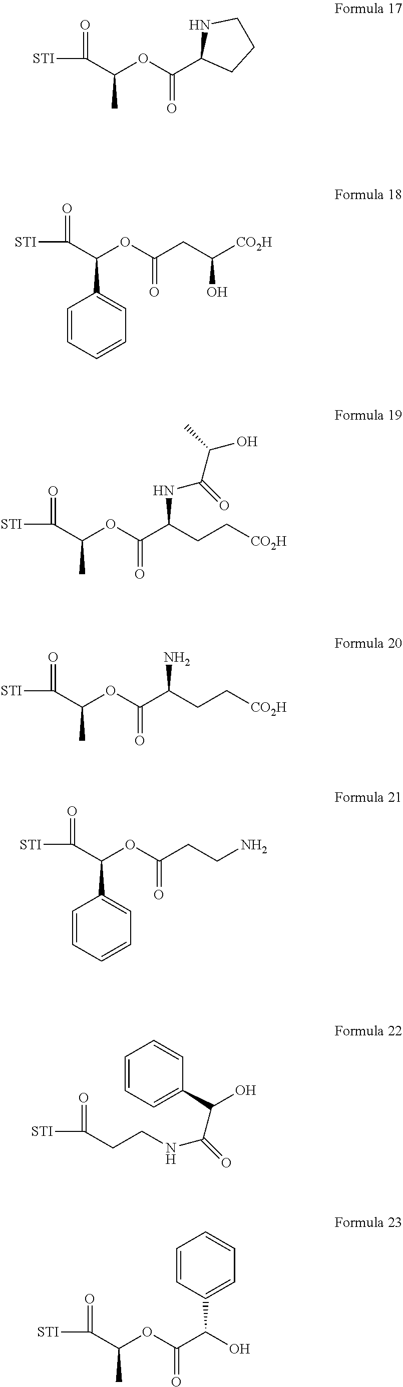

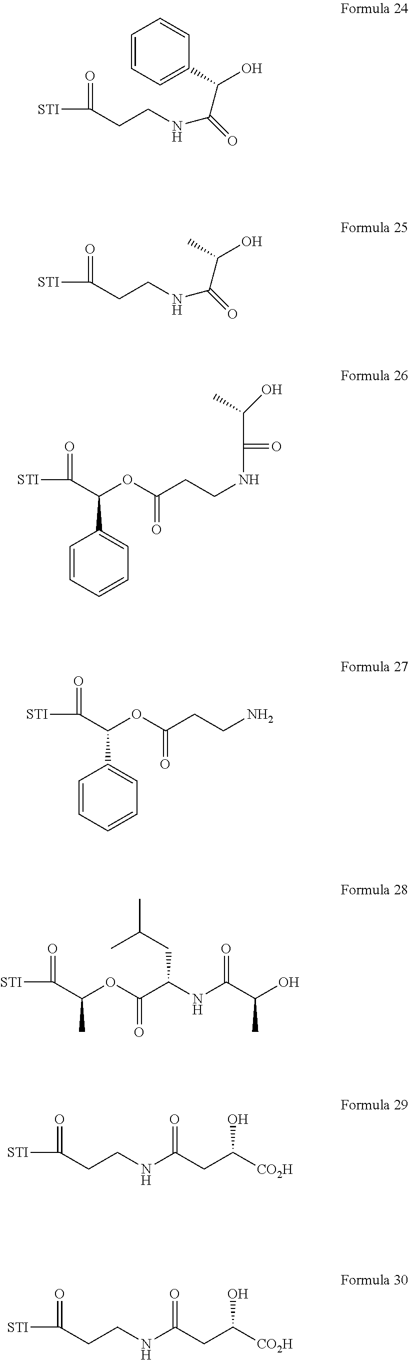

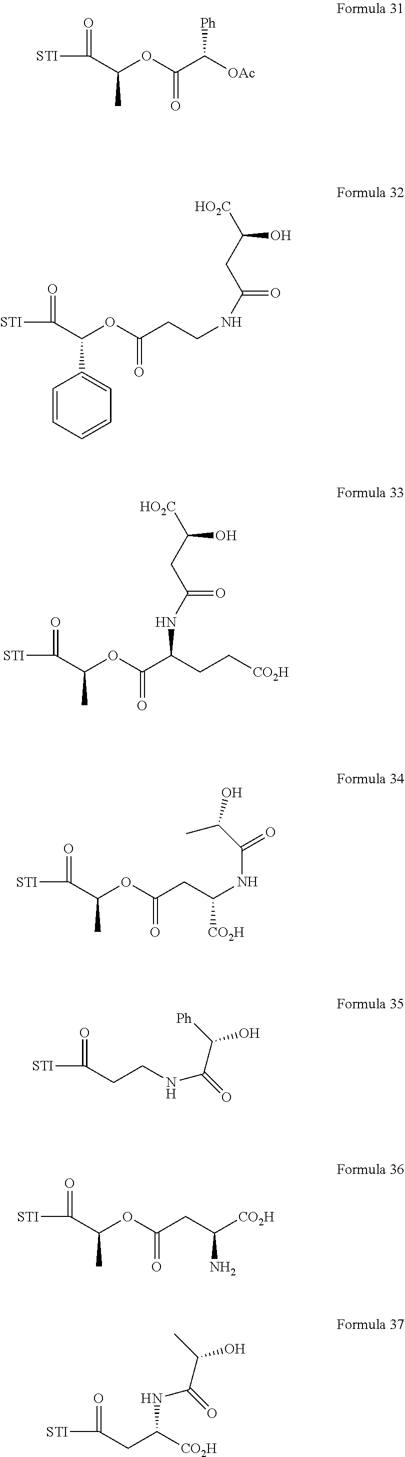

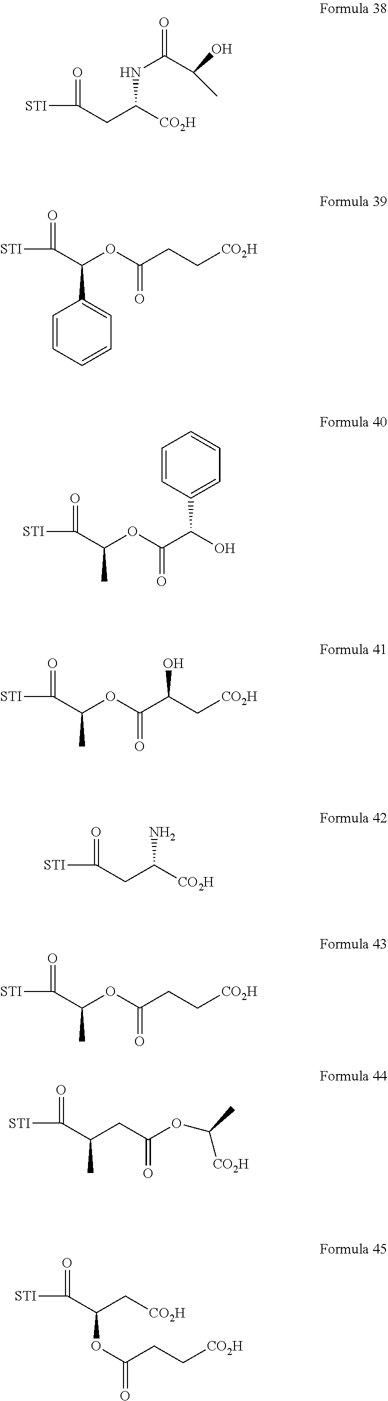

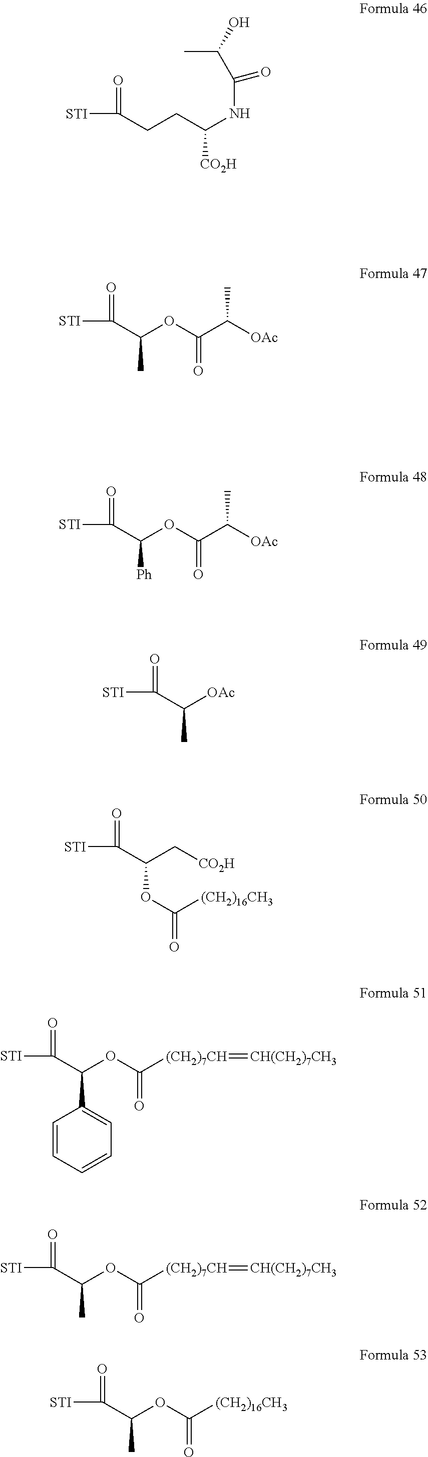

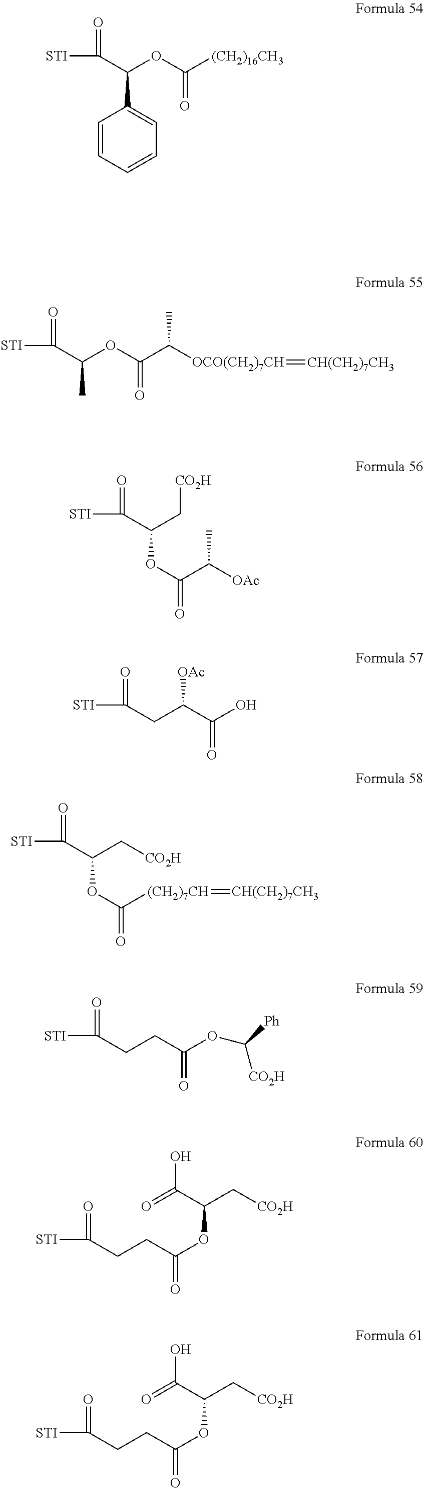

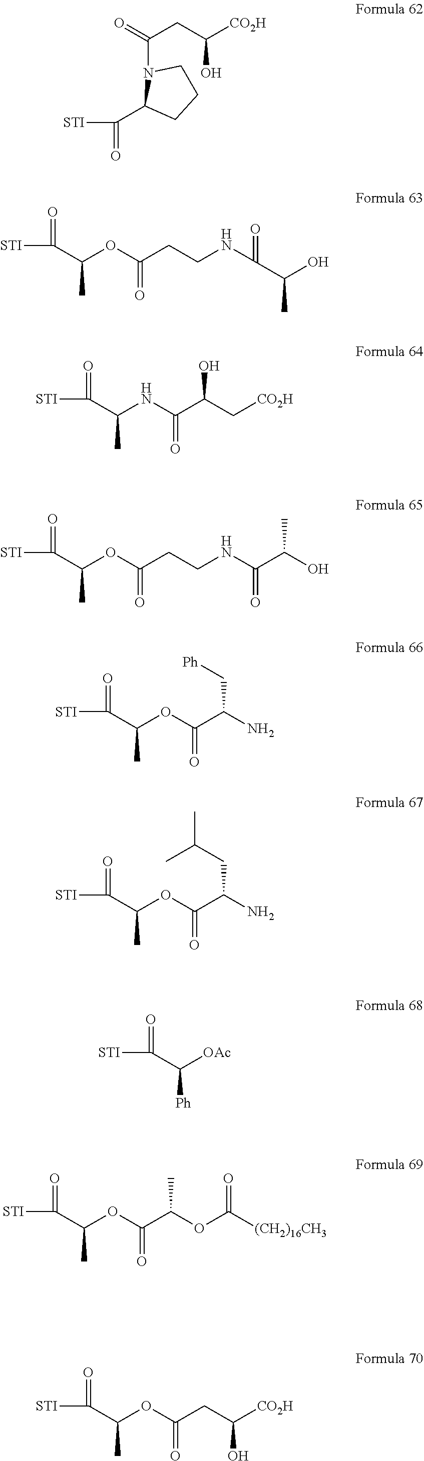

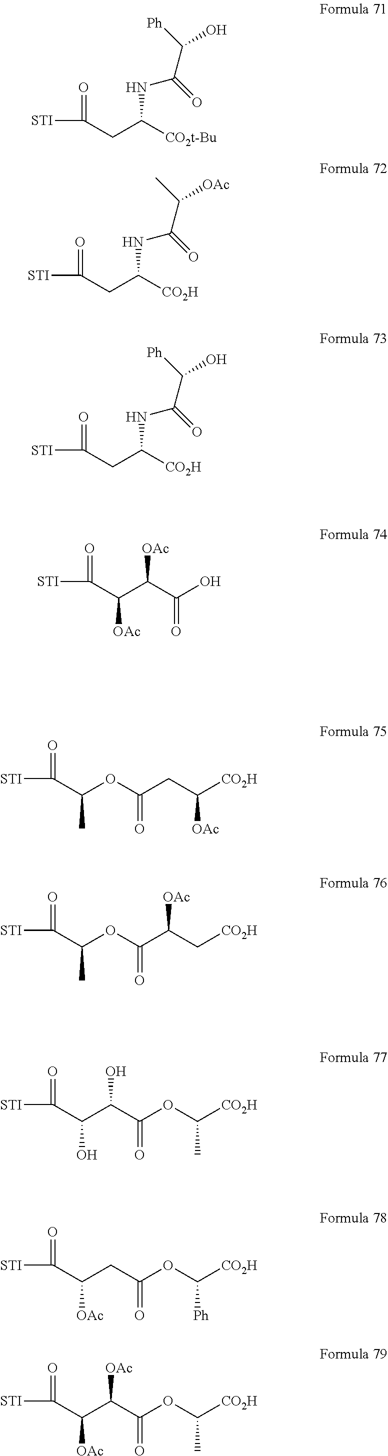

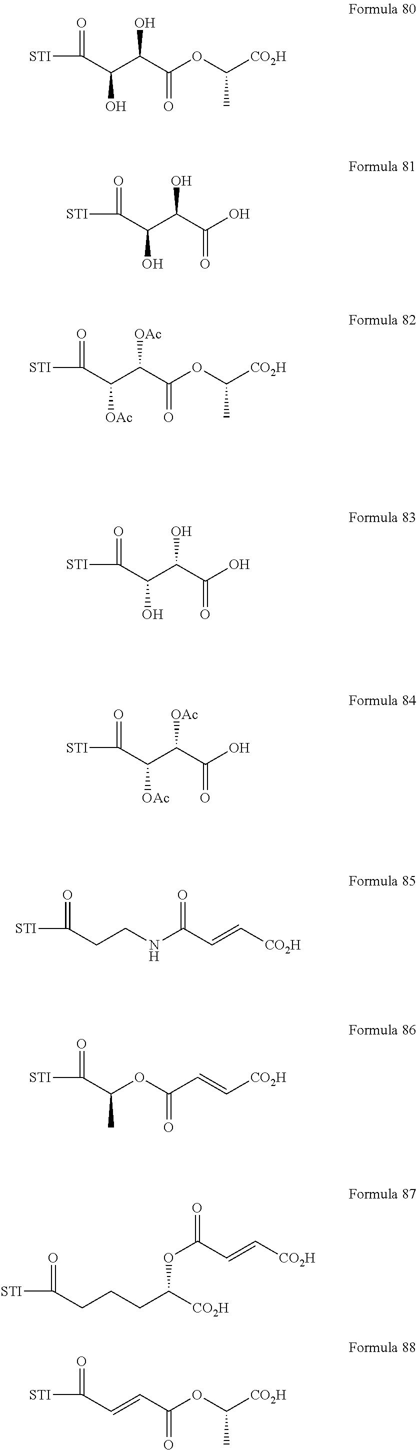

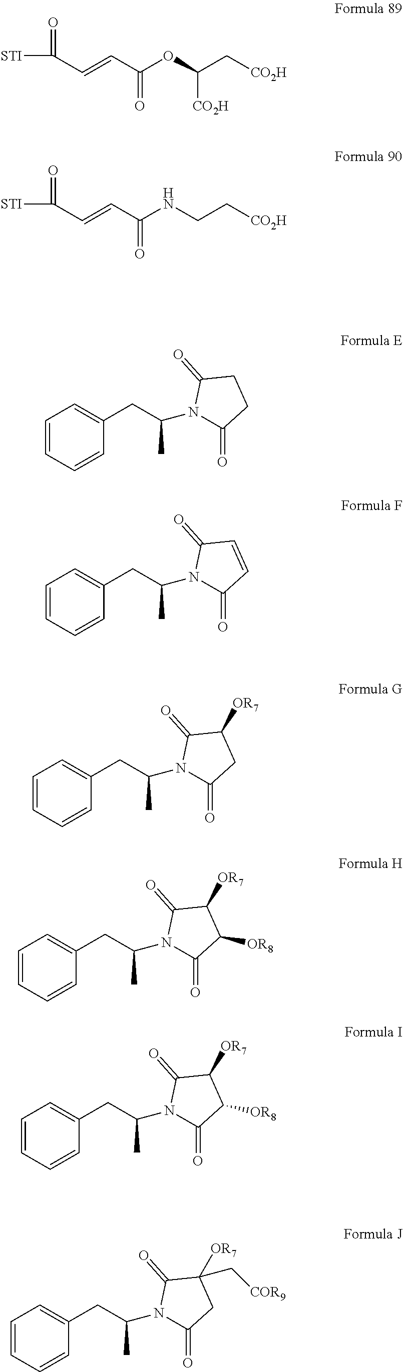

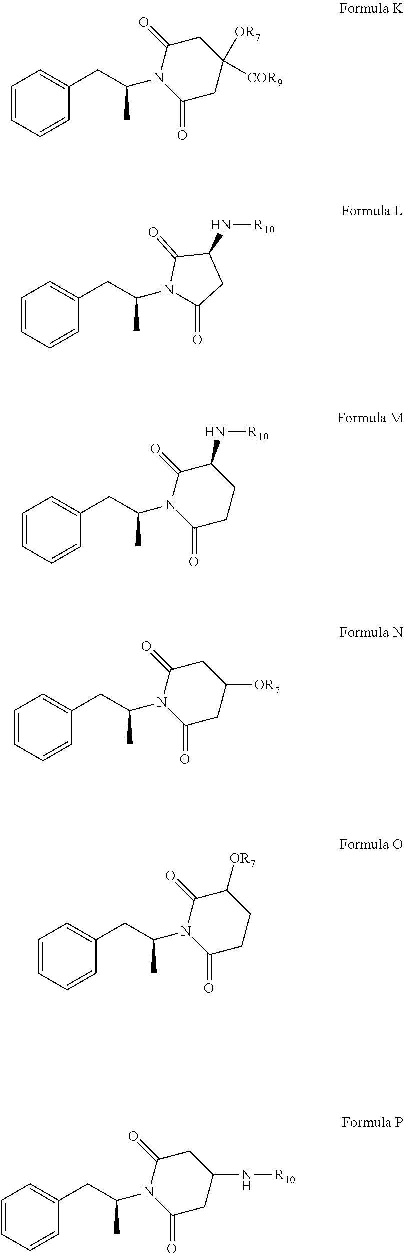

### EXAMPLES

### Processes for Preparing Stimulant Conjugates

Two general procedures may be used for the preparation of various stimulant prodrug conjugates.

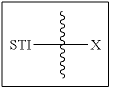

And more specifically,

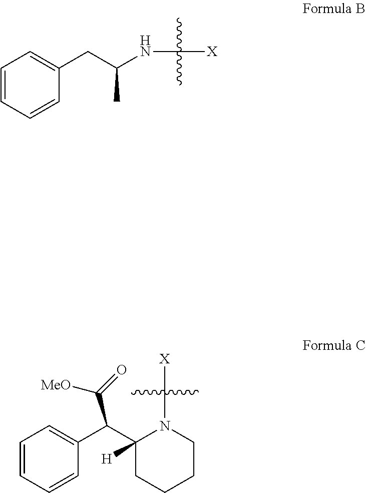

The procedure involves treating the stimulant first with a base followed by reacting the carboxyl-activated prodrug moieties.

### General Procedure 1:

Stimulant Coupling with the Activated Prodrug Side Chain; (Using Amines (TEA, DIPEA, NMM) as base procedure):

To a solution of Boc-hydroxyl protected Osu-active ester of alpha-hydroxy acid (1.05 g, 3.4 mmol) in THF (30 mL) is added d-amphetamine sulfate (1.0 eq) and TEA (4.0 eq). The resulting mixture is allowed to stir for 20 h at 20° C. Water (10 mL) is added, and the solution is stirred for 10 minutes prior to removing solvents under reduced pressure. The crude product is dissolved in EtOAc (100 mL) and washed with 2% (aq) AcOH (3×100 mL), saturated NaHCO₃ solution (2×50 mL), and brine (1×100 mL). The organic extract is dried over MgSO₄, filtered, and evaporated to dryness to afford the protected amphetamine conjugate. This intermediate can be used as is for the next deprotection step or it can be purified by either chromatography or crystallization.

The product may be further characterized by nuclear magnetic resonance (NMR) spectroscopy, mass spectroscopy (MS), and elemental analysis.

This procedure works well with any of the amine bases such as triethylamine (TEA), diisopropyl ethylamine (DIPEA), n-methyl morpholine (NMM).

### General Procedure 2:

Stimulant Coupling with the Activated Prodrug Side Chain; (KO^{t}Bu Procedure):

To a solution of stimulant (1.05 g, 3.2 mmol) in THF (10 mL) is added KO^{t}Bu (1M solution in THF, 1.05 eq.) at 0° C., then stirred at ambient temperature for 30 min. The brown solution is cooled down to −78° C. and a solution of Boc-hydroxyl protected Osu-active ester of alpha-hydroxy acid (1.05g, 3.4 mmol) in THF (20 mL) is added over a period of 5 mins. After stirring the reaction at −78° C. for 30 minutes, it is allowed to warm to RT over a period of 3 hrs. The turbid reaction mixture is poured into saturated (satd) NH₄Cl solution (150 mL), stirred for 5 mins and extracted with EtOAc (250 mL). The organic part is washed with aqueous (aq) NH₄Cl, aq. NaHCO₃, brine, dried over Na₂SO₄ and evaporated to dryness to give the product (1.5 g, purity 96.5%).

In some cases the crude product may require further purification by standard column chromatography.

The product may be further characterized by nuclear magnetic resonance (NMR) spectroscopy, mass spectroscopy (MS), and elemental analysis.

### General Procedure 3:

Stimulant Coupling with the Activated Prodrug Side Chain; (LHMDS [Lithium Hexamethyl Disilaside] Procedure):

To a solution of stimulant (1.05 g, 3.2 mmol) in THF (10 mL) is added LHMDS (LiN(TMS)₂) (1M solution in THF, 1.05 eq.) at 0° C., then stirred at ambient temperature for 30 min. The brown solution is cooled down to −78° C. and a solution of Boc-hydroxyl protected Osu-active ester of alpha-hydroxy acid (1.05 g, 3.4 mmol) in THF (20 mL) is added over a period of 5 mins. After stirring the reaction at −78° C. for 30 minutes, it is allowed to warm to room temperature (RT) over a period of 3 hrs. The turbid reaction mixture is poured into satd NH₄Cl solution (150 mL), stirred for 5 mins and extracted with EtOAc (250 mL). The organic part is washed with aqueous (aq) NH₄Cl, aq. NaHCO₃, brine, dried over Na₂SO₄, and evaporated to dryness to yield the product (1.4 g, purity 95%).

In some cases the crude product may require further purification by standard column chromatography

The product may be further characterized by NMR, MS and elemental analysis.

Boc group deprotection from the coupled stimulant prodrug or alcohol-ester product:

Boc group protection is used to protect the hydroxyl group(s) of the alpha-hydroxy carboxylic acids. After the stimulant coupling, the Boc group is removed by the following general procedure.

To a solution of the hydroxyl Boc-protected coupled product (1.5 g) in IPAc (15 mL) is added 4N HCl/dioxane (15 mL) and the reaction mixture is stirred at RT for 3 h (white ppt formation takes place after 10-15 mins). The solution is diluted with IPAc 50 mL), stirred for 10 mins. The precipitate is filtered, washed with IPAc and dried to give the deprotected product (quantitative yield). In this case the product is isolated as the HCl salt. HPLC purity ˜95%. The product may be further characterized by NMR, MS and elemental analysis.

Boc Group Deprotection from the Coupled Stimulant Prodrug Product (Another General Procedure):

Another general procedure is also used to remove the Boc group from the coupled stimulant prodrug product. To a solution of the above, hydroxyl Boc-protected coupled product (1.5 g) in dichloromethane (15 mL) is added trifluoro acetic acid (15 mL) and the reaction mixture is stirred at RT for 3 hrs. The reaction mixture is concentrated to a dry powder on a rotavap and the residue is further purified by either trituration or chromatography as a TFA salt of the enol ester prodrug product. In this case the product is isolated as the TFA salt. HPLC purity ˜95%. The product is further characterized by NMR, MS and elemental analysis.

### Synthesis of the Activated Side Chain -OSu Ester for Stimulant Coupling:

Generally, N-hydroxy succinimide ester activated carboxylic acid of the alpha-hydroxy carboxylic acid is used for stimulant coupling. To a solution of the hydroxyl Boc-protected alpha-hydroxy carboxylic acid (1 g, 1.1 mmol) and NHS (N-hydroxy succinimide) (1.05 eq) in THF (10 mL) is added a solution of DCC (1.05 eq) in THF (5 mL) at 0° C. The reaction mixture is slowly brought to RT and left overnight at RT. The turbid solution is filtered and the filtrate is used as such for the next step coupling process.

Depending on the stability of the specific compound, the -OSu ester also can be precipitated and crystallized.

### EMBODIMENTS

Various embodiments are listed below. It will be understood that the embodiments listed below may be combined with all aspects and other embodiments in accordance with the scope of the invention.

Embodiment 1: CNS Stimulant prodrugs of the following formulae where the prodrug moiety X is attached covalently to the stimulant molecule as an amide,

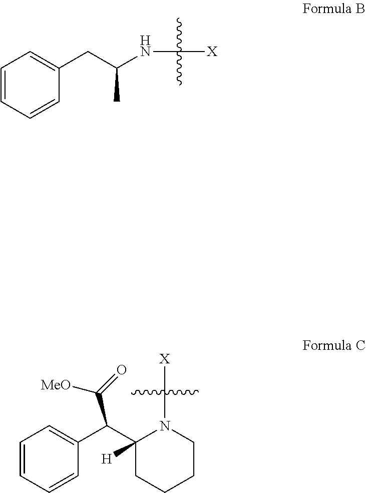

or a pharmaceutically acceptable salt thereof.

Embodiment 2: Stimulant prodrug of the following formula,

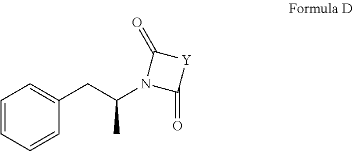

or a pharmaceutically acceptable salt thereof.

Embodiment 3: Stimulant prodrugs of embodiments 1 and 2 wherein the stimulants are amphetamine and methylphenidate.

Embodiment 4: Stimulant prodrugs of embodiment 1 wherein the prodrug moiety X is chemically/covalently attached to the amine group of amphetamine and methylphenidate as the amide.

Embodiment 5: Stimulant prodrugs of embodiment 2 wherein “Y” is part of the ligand to form the prodrug moiety as a five-membered or six-membered imide ring formed around the amphetamine amine, wherein,
Y=(CH2)t, CH═CH, CH2—CH(OR7), CH(OR7)—CH(OR8), CH2C(OR7)(CH2COR9), CH2—C(OR7)(COR9)—CH2, CH2—CH(OR7)—CH2, CH2—CH2—CH(OR7), CH2—CH(NR10), CH2—CH2—CH(NR10), or CH2—CH(NR10)—CH2, and t is an integer selected from 0 to 4.

### Wherein,

R7, R8 are each independently H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R9=OH, an ester formed by the hydroxyl group of another alpha-hydroxy acid, or an amide formed by the amine group of an amino acid, and,
R10=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid.

Embodiment 6: The stimulant prodrugs of embodiment 1, wherein X is a prodrug moiety selected from alpha-hydroxy carboxylic acid and derivatives as monomers, alpha-hydroxy carboxylic acid homo-oligomers, alpha-hydroxy carboxylic acid hetero oligomers with another alpha-hydroxy carboxylic acid, alpha-hydroxy carboxylic acid hetero oligomers with amino acids, alpha-hydroxy carboxylic acid hetero oligomers with dicarboxylic acids, alpha-hydroxy carboxylic acid hetero oligomers with fatty acids, fatty acids, and other GRAS-based reagents.

Embodiment 7: The stimulant prodrugs of embodiment 6 wherein homo- and hetero-‘mers’ include both linear and branched ‘mers’. The homo- and hetero-‘mers’ may also be cross linked with other GRAS reagents such as alpha-hydroxy carboxylic acid and amino acids.

Embodiment 8: The stimulant prodrugs of embodiment 6 wherein the alpha-hydroxy carboxylic acid is lactic acid, tartaric acid, malic acid, citric acid, mandelic acid, pantoic acid, pantothenic acid, 2-hydroxy glutaric acid, 3-hydroxy glutaric acid, and other poly-hydroxy carboxylic acids derived from sugars and carbohydrates. The naturally occurring (L)-isomers, the non-natural (D)-isomers, varying mixtures of (L) and (D) isomers, racemates and mixtures of diastereomers, and meso-isomers are all claimed in this invention.

Embodiment 9: The stimulant prodrugs of embodiment 6 wherein the amino acids represented here include both natural (all 22 of the proteinogenic amino acids), and non-natural amino acids, the naturally occurring (L)-isomers, the non-natural (D)-isomers, varying mixtures of (L) and (D) isomers, racemates and mixtures of diastereomers. The amino acids represented here also include alpha amino acids, beta amino acids, gamma amino acids, and epsilon amino acids (amino group remote relative to the carboxyl group).

Embodiment 10: The stimulant prodrugs of embodiment 6 wherein the fatty acids represented here are long chain carboxylic acids, ranging in lengths between eight carbons (C8) to twenty carbons (C20), and said fatty acids are linear or branched chains, and either saturated or unsaturated chains, and in the case of unsaturated fatty acids includes both cis-and trans-isomers (Z and E isomers), wherein examples of such fatty acids include, but are not limited to, sorbic acid, stearic acid, oleic acid, palmitic acid, and linoleic acid.

Embodiment 11: The stimulant prodrugs of embodiment 6 wherein the dicarboxylic acids represented here to make hetero oligomers with alpha-hydroxy carboxylic acid include, but not limited to, fumaric acid, maleic acid, and succinic acid.

Embodiment 12: The stimulant prodrugs of embodiment 2 wherein, when “Y” is part of a five-membered imide ring formed with amphetamine nitrogen, for example: succinimide (formula E), maleiimide (formula F), malic acid imide (formula G, both isomers), tartaric acid imide (formula H, formula I, RR, SS, and meso-isomers), citric acid imide (formula J), or aspartic acid imide (formula L).

Embodiment 13: The stimulant prodrugs of embodiment 2 wherein, when “Y” is part of a six-membered imide ring formed with amphetamine nitrogen, for example: citric acid imide (formula K), glutamic acid imide (formula M), 2-hydroxy glutaric acid imide (formula O), 3-hydroxy glutaric acid imide (formula N), or 3-amino glutaric acid imide (formula P).

Embodiment 14: The stimulant prodrugs of embodiment 1, wherein ligand X is further represented as any of ligands 1-15 (shown below);

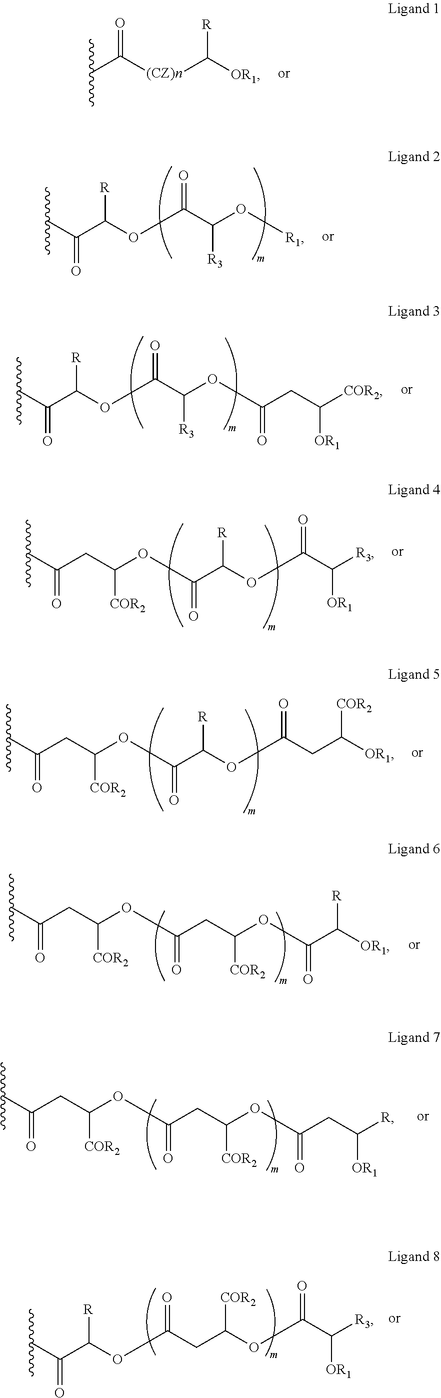

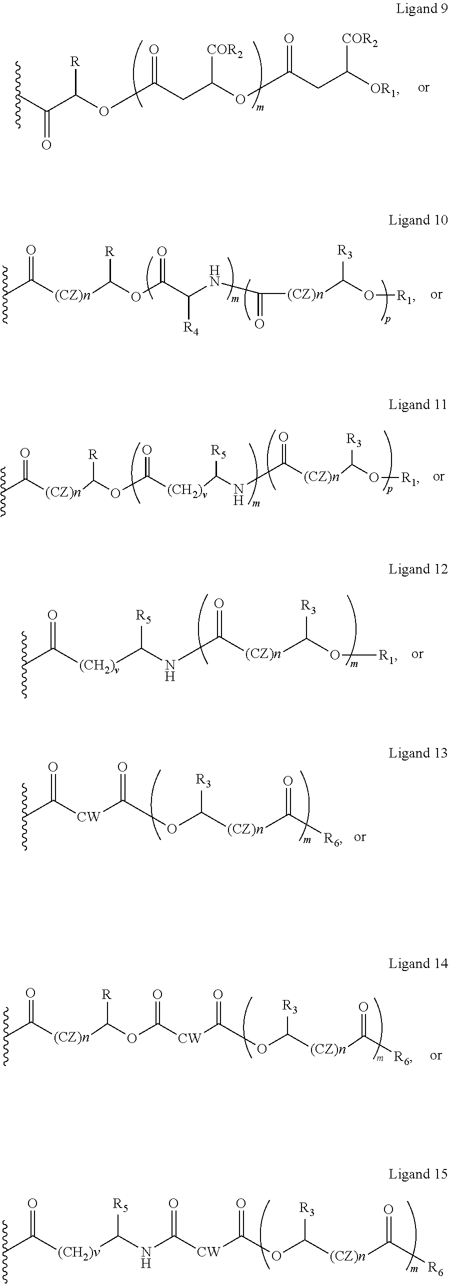

Wherein, in ligands 1-15,
CZ=CH2, or CHOR1,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid,
R=Me, Ph, CH2COR2, CHOR1COR2, or COR2 (when n is not zero),
R2=OH, or is an ester formed by the hydroxyl group of another alpha-hydroxy acid or is an amide formed by the amine group of a natural or non-natural amino acid, including (L)-isomers, (D)-isomers, mixtures of (L) and (D) isomers, racemates and mixtures of diastereomers, or is O-alkyl (alkyl esters, where the alkyl group is 1-4 carbon linear or branched, saturated or non-saturated alkyl groups),
R3=Me, Ph, CH2COR2, CHOR1COR2, or COR2 (when n is not zero),
R4 is the side chain of a natural or non-natural amino acid, including side chains of (L)-isomers, (D)-isomers, mixtures of (L) and (D) isomers, racemates and mixtures of diastereomers, and the amino acids represented here depicts both natural and non-natural amino acids, the naturally occurring (L)-isomers, the non-natural (D)-isomers, mixtures of (L) and (D) isomers, racemates and mixtures of diastereomers (R4 in ligands 10 and 12),
R5=H, or COR2,
CW=(CH2)q, or CH═CH (both E and Z isomers),
R6=OH or is an ester formed by the hydroxyl group of another alpha-hydroxy acid or is an amide formed by the amine group of an amino acid, or an alkyl ester, and the amino acids include both natural and non-natural amino acids, (L)-isomers, (D)-isomers, mixtures of (L) and (D) isomers, racemates and mixtures of diastereomers, or R6 is an ester with an alkyl group (O-alkyl, alkyl group is 1-4 carbon linear or branched, saturated or non-saturated alkyl groups),
and m is an integer selected from 0 to 4, and n is an integer selected from 0 to 2, and q is an integer selected from 2 to 6, and v is an integer selected from 0 to 6

Embodiment 15: The stimulant prodrugs of embodiment 1, wherein X is a prodrug moiety and is represented by ligand 16;

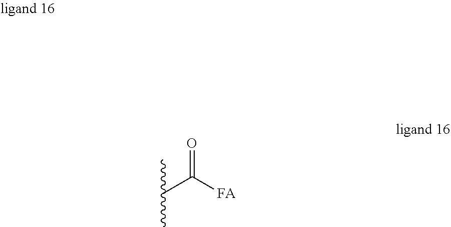

### Wherein,

FA is C8 to C20 saturated fatty acids, or C8 to C20 unsaturated fatty acids, including but not limited to, sorbic acid, stearic acid, oleic acid, palmitic acid, linoleic acid. These fatty acids could be either linear or branched chain fatty acids, and in the case of unsaturated fatty acids, either cis- or trans-isomers (Z and E isomers).

Embodiment 16: Stimulant prodrug compounds represented by one of formulae 1-90 or any one of formulae E-P.

Embodiment 17: A composition comprising the compound of any of embodiments 1-16

Embodiment 18: The composition of embodiment 16 wherein the compound or pharmaceutically acceptable salts thereof maintains a steady-state release curve in blood that provides therapeutically effective stimulant bioavailability.

Embodiment 19: The composition of embodiment 17, wherein when said composition is administered orally and the bioavailability of stimulant is maintained.

Embodiment 20: A method of treating CNS diseases comprising orally administering the composition of embodiment 18 to a patient.

Embodiment 21: The pharmaceutical composition of embodiment 17, wherein the said composition is a pharmaceutically acceptable salt form.

Embodiment 22: A pharmaceutical composition comprising one or more of the stimulant prodrugs of embodiment 17 and one or more pharmaceutically acceptable excipients.

Embodiment 23: Stimulant prodrugs of Embodiment 17 wherein the stimulant are amphetamine and methylphenidate.

Embodiment 24: Stimulant prodrugs of Embodiment 23 wherein the stimulants amphetamine and methylphenidate can have any stereogenic configuration, including both dextro- and levo-isomers, racemates, and mixtures of the two isomers of varying ratios. The dextro-isomers, particularly dextroamphetamine, and dextro-methylphenidate are preferred.

1. A stimulant prodrug of an amphetamine compound where a prodrug moiety X is attached covalently to an amine of the amphetamine compound as an amide, or a pharmaceutically acceptable salt thereof.

2. A stimulant prodrug of an amphetamine compound where a prodrug moiety Y is attached covalently to an amine of the amphetamine compound as a five-membered or six-membered imide ring, or a pharmaceutically acceptable salt thereof.

3. The stimulant prodrug of claim 1 wherein the amphetamine compound is amphetamine or methylphenidate.

4. The stimulant prodrug of claim 2 wherein the amphetamine compound is amphetamine.

5. The stimulant prodrug of claim 2 wherein “Y” is selected from the group consisting of Y=(CH2)t, CH═CH, CH2—CH(OR7), CH(OR7)—CH(OR8), CH2C(OR7)(CH2COR9), CH2—C(OR7)(COR9)—CH2, CH2—CH(OR7)—CH2, CH2—CH2—CH(OR7), CH2—CH(NR10), CH2—CH2—CH(NR10), and CH2—CH(NR10)—CH2, and t is an integer selected from 0 to 4,
wherein,
R7, R8 are each independently H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid, and,
R9=OH, an ester formed by the hydroxyl group of another alpha-hydroxy acid, or an amide formed by the amine group of an amino acid, and,
R10=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid.

6. The stimulant prodrug according to claim 1 wherein X is selected from the group consisting of alpha-hydroxy carboxylic acid and derivatives as monomers, alpha-hydroxy carboxylic acid homo-oligomers, alpha-hydroxy carboxylic acid hetero oligomers with another alpha-hydroxy carboxylic acid, alpha-hydroxy carboxylic acid hetero oligomers with amino acid, alpha-hydroxy carboxylic acid hetero oligomers with dicarboxylic acids, alpha-hydroxy carboxylic acid hetero oligomers with fatty acids, fatty acids, and other GRAS-based reagents.

7. The stimulant prodrug according to claim 6 wherein homo- and hetero-‘mers’ are linear or branched ‘mers’ wherein the hetero-‘mers’ are cross linked with other GRAS reagents, wherein the alpha-hydroxy carboxylic acid is selected from the group consisting of lactic acid, tartaric acid, malic acid, citric acid, mandelic acid, pantoic acid, pantothenic acid, 2-hydroxy glutaric acid, 3-hydroxy glutaric acid, and other poly-hydroxy carboxylic acids derived from sugars and carbohydrates, wherein the amino acids are selected from the group consisting of naturally occurring proteinogenic amino acids, non-natural amino acids, (L)-isomers, (D)-isomers, mixtures of the (L) and (D) isomers, racemates and mixtures of diastereomers, wherein the fatty acids are long chain carboxylic acids, ranging in lengths from eight carbons (C8) to twenty carbons (C20), and wherein the dicarboxylic acids of the hetero oligomers with alpha-hydroxy carboxylic acid are fumaric acid, maleic acid, or succinic acid.

8. (canceled)

9. (canceled)

10. (canceled)

11. The stimulant prodrug according to claim 7 wherein the fatty acid is sorbic acid, stearic acid, oleic acid, palmitic acid, or linoleic acid.

12. (canceled)

13. The stimulant prodrug of claim 2 wherein “Y” is part of a five-membered imide ring selected from succinimide (formula E), malei-imide (formula F). malic acid imide (formula G, both isomers), tartaric acid imide (formula H, formula I, RR, SS, and meso-isomers), citric acid imide (formula J), and aspartic acid imide (formula L).

14. The stimulant prodrug of claim 2 wherein “Y” is part of a six-membered imide ring selected from citric acid imide (formula K), glutamic acid imide (formula M), 2-hydroxy glutaric acid imide (formula O), 3-hydroxy glutaric acid imide (formula N), and 3-amino glutaric acid imide (formula P).

15. The stimulant prodrug according to claim 1 wherein X is selected from any of ligands 1-15: 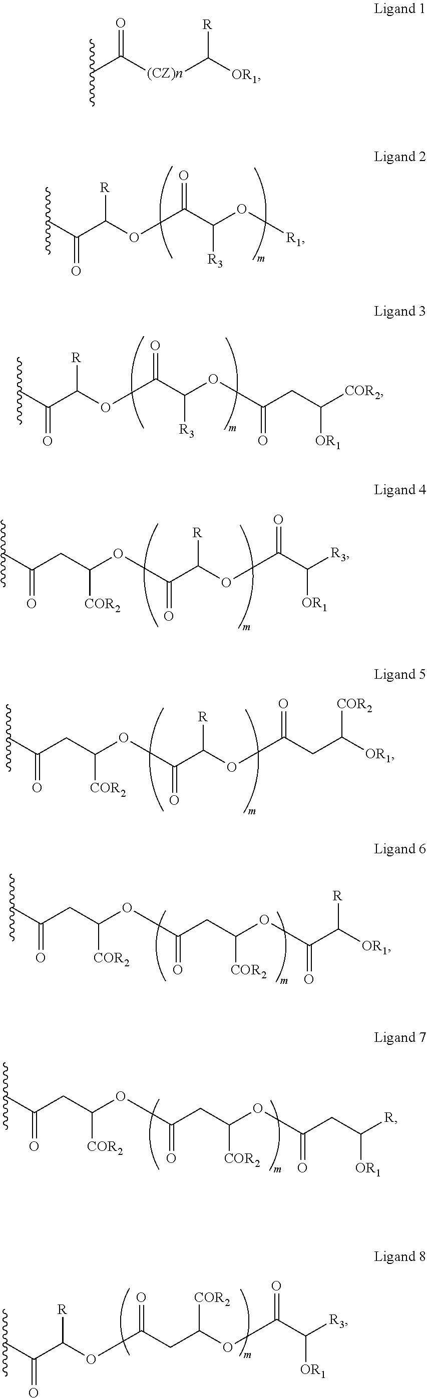 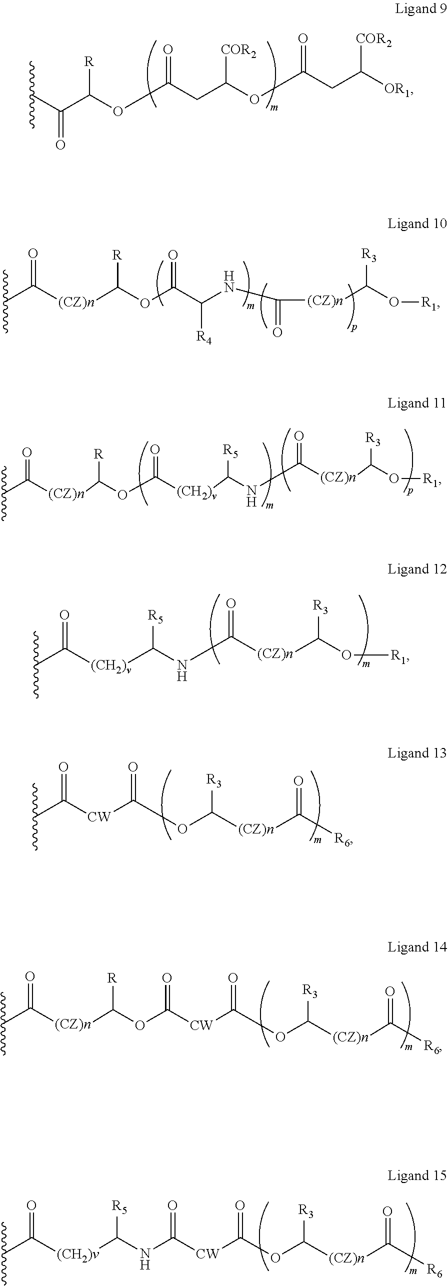
wherein, in ligands 1-15,
CZ=CH2, or CHOR1,
R1=H, an acyl linkage of a fatty acid, an acyl linkage of an alpha-hydroxy acid, an acyl linkage of an amino acid, or an acyl linkage of a dicarboxylic acid including, but not limited to, fumaric acid, maleic acid and succinic acid,
R=Me, Ph, CH2COR2, CHOR1COR2, or COR2 (when n is not zero),
R2=OH, or is an ester formed by the hydroxyl group of another alpha-hydroxy acid or is an amide formed by the amine group of a natural or non-natural amino acid, including (L)-isomers, (D)-isomers, mixtures of (L) and (D) isomers, racemates and mixtures of diastereomers, or is O-alkyl (alkyl ester, where the alkyl group is 1-4 carbon linear or branched, saturated or non-saturated alkyl groups),
R3=Me, Ph, CH2COR2, CHOR1COR2, or COR2 (when n is not zero),
R4 is the side chain of a natural or non-natural amino acid, including side chains of (L)-isomers, (D)-isomers, mixtures of (L) and (D) isomers, racemates and mixtures of diastereomers, (R4 in ligands 10 and 12),
R5=H, or COR2,
CW=(CH2)q, or CH═CH (both E and Z isomers),
R6=OH or is an ester formed by the hydroxyl group of another alpha-hydroxy acid or is an amide formed by the amine group of an amino acid, or alkyl ester, wherein the amino acids are natural or non-natural amino acids, (L)-isomers, (D)-isomers, mixtures of (L) and (D) isomers, racemates or mixtures of diastereomers, or R6 is an ester with an alkyl group (O-alkyl, alkyl group is 1-4 carbon linear and branched, saturated and non-saturated alkyl groups),
and m is an integer selected from 0 to 4, and n is an integer selected from 0 to 2, and q is an integer selected from 2 to 6, and v is an integer selected from 0 to 6.

16. The stimulant prodrug according to claim 1 wherein X is represented by ligand 16: 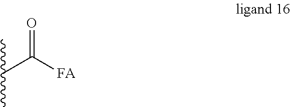
wherein,
FA is C8 to C20 saturated or unsaturated, linear or branched, fatty acid.

17. The stimulant prodrug according to claim 16, wherein FA is sorbic acid, stearic acid, oleic acid, palmitic acid, or linoleic acid.

18. An amphetamine compound prodrug represented by any one of formulae 1-90 or any one of formulae E-P.

19. The prodrug of claim 18 wherein the amphetamine compound is amphetamine or methylphenidate.

20. The prodrug of claim 19 wherein the amphetamine compound is a dextro- or levo-isomer, a racemate, or a mixture of two isomers of varying ratios.

21. A pharmaceutical composition comprising compound of claim 1, and a pharmaceutically acceptable excipient.

22. (canceled)

23. The pharmaceutical composition of claim 21, wherein the compound is a pharmaceutically acceptable salt form.

24. A method of treating CNS diseases comprising orally administering to a patient in need thereof the pharmaceutical composition of claim 21.

25. The prodrug of claim 20, wherein the dextro-isomer is dextroamphetamine or dextro-methylphenidate.

26. (canceled)

27. (canceled)
